SEQUENCE LISTING (27 sequences)

SEQ ID NO: 1 - DNA, 723 bp, Aequorea victoria
  source 1..723: mol_type = other DNA; note = fuGFP Wild types
atggtgagcagtggcgaagatattttctcgggcttggttccgattctgatcgagctggagggcgatgtgaacggtcatcgttttagcgttcgcggtgaaggttatggcgacgcgagcaacggcaaactggaaattaagttcatctgcacgaccggtcgcctgccggtgccttggccgaccttggtgacgaccttgtcgtatggcgtgcagtgttttgcgaagtatccggagcacatgcgccaaaacgatttctttaaaagtgcgatgccggacggttacgtccaggagcgtaccatttccttcaaggaagatggcacgtacaaaactcgcgcagaggttaagtttgaaggtgaagcgctggtcaatcgtatcgatttgaagggtttggagtttaaagaggatggtaacattctgggccataaactggagtatagcttcaacagccattatgtttacattacggcagacaagaatcgtaacggcttggaggcccaattccgtattcgccacaatgttgatgacggtagcgtccaactggccgaccattaccaacagaacaccccaattggtgagggtccggtgttgctgccggaacaacactatctgacgaccaatagcgtgctgagcaaagatccgcaggaacgccgtgatcacatggtcctggtggaatttgtgaccgctgcgggcttgagcctgggtatggacgagctgtataagagctaa

SEQ ID NO: 2 - protein, 240 aa, Aequorea victoria
  source 1..240: note = fuGFP Wild Type
MVSSGEDIFSGLVPILIELEGDVNGHRFSVRGEGYGDASNGKLEIKFICTTGRLPVPWPTLVTTLSYGVQCFAKYPEHMRQNDFFKSAMPDGYVQERTISFKEDGTYKTRAEVKFEGEALVNRIDLKGLEFKEDGNILGHKLEYSFNSHYVYITADKNRNGLEAQFRIRHNVDDGSVQLADHYQQNTPIGEGPVLLPEQHYLTTNSVLSKDPQERRDHMVLVEFVTAAGLSLGMDELYKS

SEQ ID NO: 3 - protein, 238 aa, Aequorea victoria
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 4 - DNA, 765 bp, Aequorea victoria
  source 1..765: mol_type = other DNA; note = DNA sequence of N-terminal 6xHis tag fuGFP (NHfuGFP Wild Type)
atgggtcatcaccatcatcatcacggaagcggttctagcggtatggtgagcagtggcgaagatattttctcgggcttggttccgattctgatcgagctggagggcgatgtgaacggtcatcgttttagcgttcgcggtgaaggttatggcgacgcgagcaacggcaaactggaaattaagttcatctgcacgaccggtcgcctgccggtgccttggccgaccttggtgacgaccttgtcgtatggcgtgcagtgttttgcgaagtatccggagcacatgcgccaaaacgatttctttaaaagtgcgatgccggacggttacgtccaggagcgtaccatttccttcaaggaagatggcacgtacaaaactcgcgcagaggttaagtttgaaggtgaagcgctggtcaatcgtatcgatttgaagggtttggagtttaaagaggatggtaacattctgggccataaactggagtatagcttcaacagccattatgtttacattacggcagacaagaatcgtaacggcttggaggcccaattccgtattcgccacaatgttgatgacggtagcgtccaactggccgaccattaccaacagaacaccccaattggtgagggtccggtgttgctgccggaacaacactatctgacgaccaatagcgtgctgagcaaagatccgcaggaacgccgtgatcacatggtcctggtggaatttgtgaccgctgcgggcttgagcctgggtatggacgagctgtataagagctaa

SEQ ID NO: 5 - protein, 254 aa, Aequorea victoria
  source 1..254: note = protein sequence of N-terminal 6xHis tag fuGFP (NHfuGFP Wild Type)
MGHHHHHHGSGSSGMVSSGEDIFSGLVPILIELEGDVNGHRFSVRGEGYGDASNGKLEIKFICTTGRLPVPWPTLVTTLSYGVQCFAKYPEHMRQNDFFKSAMPDGYVQERTISFKEDGTYKTRAEVKFEGEALVNRIDLKGLEFKEDGNILGHKLEYSFNSHYVYITADKNRNGLEAQFRIRHNVDDGSVQLADHYQQNTPIGEGPVLLPEQHYLTTNSVLSKDPQERRDHMVLVEFVTAAGLSLGMDELYKS

SEQ ID NO: 6 - DNA, 765 bp, Aequorea victoria
  source 1..765: mol_type = other DNA; note = DNA sequence of N-terminal 6xHis tag fuGFP NHfuGFP G68S
atgggtcatcaccatcatcatcacggaagcggttctagcggtatggtgagcagtggcgaagatattttctcgggcttggttccgattctgatcgagctggagggcgatgtgaacggtcatcgttttagcgttcgcggtgaaggttatggcgacgcgagcaacggcaaactggaaattaagttcatctgcacgaccggtcgcctgccggtgccttggccgaccttggtgacgaccttgtcgtattctgtgcagtgttttgcgaagtatccggagcacatgcgccaaaacgatttctttaaaagtgcgatgccggacggttacgtccaggagcgtaccatttccttcaaggaagatggcacgtacaaaactcgcgcagaggttaagtttgaaggtgaagcgctggtcaatcgtatcgatttgaagggtttggagtttaaagaggatggtaacattctgggccataaactggagtatagcttcaacagccattatgtttacattacggcagacaagaatcgtaacggcttggaggcccaattccgtattcgccacaatgttgatgacggtagcgtccaactggccgaccattaccaacagaacaccccaattggtgagggtccggtgttgctgccggaacaacactatctgacgaccaatagcgtgctgagcaaagatccgcaggaacgccgtgatcacatggtcctggtggaatttgtgaccgctgcgggcttgagcctgggtatggacgagctgtataagagctaa

SEQ ID NO: 7 - protein, 254 aa, Aequorea victoria
  source 1..254: note = N-terminal 6xHis tag fuGFP (NHfuGFP Wild Type)
MGHHHHHHGSGSSGMVSSGEDIFSGLVPILIELEGDVNGHRFSVRGEGYGDASNGKLEIKFICTTGRLPVPWPTLVTTLSYSVQCFAKYPEHMRQNDFFKSAMPDGYVQERTISFKEDGTYKTRAEVKFEGEALVNRIDLKGLEFKEDGNILGHKLEYSFNSHYVYITADKNRNGLEAQFRIRHNVDDGSVQLADHYQQNTPIGEGPVLLPEQHYLTTNSVLSKDPQERRDHMVLVEFVTAAGLSLGMDELYKS

SEQ ID NO: 8 - DNA, 3659 bp, synthetic construct
  source 1..3659: mol_type = other DNA; note = pKaLNHfuGFPWild Type
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggattaaggaggtaaataaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaataactcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcccatatggagctcccatggtgtacacctaggagatctgcgatcgcgtttgcgatcgcgtttggaggtaaataaatgggtcatcaccatcatcatcacggaagcggttctagcggtatggtgagcagtggcgaagatattttctcgggcttggttccgattctgatcgagctggagggcgatgtgaacggtcatcgttttagcgttcgcggtgaaggttatggcgacgcgagcaacggcaaactggaaattaagttcatctgcacgaccggtcgcctgccggtgccttggccgaccttggtgacgaccttgtcgtatggcgtgcagtgttttgcgaagtatccggagcacatgcgccaaaacgatttctttaaaagtgcgatgccggacggttacgtccaggagcgtaccatttccttcaaggaagatggcacgtacaaaactcgcgcagaggttaagtttgaaggtgaagcgctggtcaatcgtatcgatttgaagggtttggagtttaaagaggatggtaacattctgggccataaactggagtatagcttcaacagccattatgtttacattacggcagacaagaatcgtaacggcttggaggcccaattccgtattcgccacaatgttgatgacggtagcgtccaactggccgaccattaccaacagaacaccccaattggtgagggtccggtgttgctgccggaacaacactatctgacgaccaatagcgtgctgagcaaagatccgcaggaacgccgtgatcacatggtcctggtggaatttgtgaccgctgcgggcttgagcctgggtatggacgagctgtataagagctaagtgactagtgctgtgactagtgctagcggcgcgccctcgagggtaccgaattcgcggccgc

SEQ ID NO: 9 - DNA, 2825 bp, synthetic construct
  source 1..2825: mol_type = other DNA; note = Complete sequence of pALNHfuGFPS68G , mutation is S68G
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcccatatggagctcccatggtgtacacctaggagatctgcgatcgcgtttgcgatcgcgtttggaggtaaataaatgggtcatcaccatcatcatcacggaagcggttctagcggtatggtgagcagtggcgaagatattttctcgggcttggttccgattctgatcgagctggagggcgatgtgaacggtcatcgttttagcgttcgcggtgaaggttatggcgacgcgagcaacggcaaactggaaattaagttcatctgcacgaccggtcgcctgccggtgccttggccgaccttggtgacgaccttgtcgtattctgtgcagtgttttgcgaagtatccggagcacatgcgccaaaacgatttctttaaaagtgcgatgccggacggttacgtccaggagcgtaccatttccttcaaggaagatggcacgtacaaaactcgcgcagaggttaagtttgaaggtgaagcgctggtcaatcgtatcgatttgaagggtttggagtttaaagaggatggtaacattctgggccataaactggagtatagcttcaacagccattatgtttacattacggcagacaagaatcgtaacggcttggaggcccaattccgtattcgccacaatgttgatgacggtagcgtccaactggccgaccattaccaacagaacaccccaattggtgagggtccggtgttgctgccggaacaacactatctgacgaccaatagcgtgctgagcaaagatccgcaggaacgccgtgatcacatggtcctggtggaatttgtgaccgctgcgggcttgagcctgggtatggacgagctgtataagagctaagtgactagtgctgtgactagtgctagcggcgcgccctcgagggtaccgaattcgcggccgc

SEQ ID NO: 10 - DNA, 3659 bp, synthetic construct
  source 1..3659: mol_type = other DNA; note = Complete sequence of pKaLfuGFPG68S, mutation is S68G
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggattaaggaggtaaataaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaataactcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcccatatggagctcccatggtgtacacctaggagatctgcgatcgcgtttgcgatcgcgtttggaggtaaataaatgggtcatcaccatcatcatcacggaagcggttctagcggtatggtgagcagtggcgaagatattttctcgggcttggttccgattctgatcgagctggagggcgatgtgaacggtcatcgttttagcgttcgcggtgaaggttatggcgacgcgagcaacggcaaactggaaattaagttcatctgcacgaccggtcgcctgccggtgccttggccgaccttggtgacgaccttgtcgtattctgtgcagtgttttgcgaagtatccggagcacatgcgccaaaacgatttctttaaaagtgcgatgccggacggttacgtccaggagcgtaccatttccttcaaggaagatggcacgtacaaaactcgcgcagaggttaagtttgaaggtgaagcgctggtcaatcgtatcgatttgaagggtttggagtttaaagaggatggtaacattctgggccataaactggagtatagcttcaacagccattatgtttacattacggcagacaagaatcgtaacggcttggaggcccaattccgtattcgccacaatgttgatgacggtagcgtccaactggccgaccattaccaacagaacaccccaattggtgagggtccggtgttgctgccggaacaacactatctgacgaccaatagcgtgctgagcaaagatccgcaggaacgccgtgatcacatggtcctggtggaatttgtgaccgctgcgggcttgagcctgggtatggacgagctgtataagagctaagtgactagtgctgtgactagtgctagcggcgcgccctcgagggtaccgaattcgcggccgc

SEQ ID NO: 11 - DNA, 2825 bp, synthetic construct
  source 1..2825: mol_type = other DNA; note = Complete sequence of pALNHfuGFPWT
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggttctcgcggtatcattgcagcactggggccagatggtaagccctcccgtatcgtagttatctacacgacggggagtcaggcaactatggatgaacgaaatagacagatcgctgagataggtgcctcactgattaagcattggtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcccatatggagctcccatggtgtacacctaggagatctgcgatcgcgtttgcgatcgcgtttggaggtaaataaatgggtcatcaccatcatcatcacggaagcggttctagcggtatggtgagcagtggcgaagatattttctcgggcttggttccgattctgatcgagctggagggcgatgtgaacggtcatcgttttagcgttcgcggtgaaggttatggcgacgcgagcaacggcaaactggaaattaagttcatctgcacgaccggtcgcctgccggtgccttggccgaccttggtgacgaccttgtcgtatggcgtgcagtgttttgcgaagtatccggagcacatgcgccaaaacgatttctttaaaagtgcgatgccggacggttacgtccaggagcgtaccatttccttcaaggaagatggcacgtacaaaactcgcgcagaggttaagtttgaaggtgaagcgctggtcaatcgtatcgatttgaagggtttggagtttaaagaggatggtaacattctgggccataaactggagtatagcttcaacagccattatgtttacattacggcagacaagaatcgtaacggcttggaggcccaattccgtattcgccacaatgttgatgacggtagcgtccaactggccgaccattaccaacagaacaccccaattggtgagggtccggtgttgctgccggaacaacactatctgacgaccaatagcgtgctgagcaaagatccgcaggaacgccgtgatcacatggtcctggtggaatttgtgaccgctgcgggcttgagcctgggtatggacgagctgtataagagctaagtgactagtgctgtgactagtgctagcggcgcgccctcgagggtaccgaattcgcggccgc

SEQ ID NO: 12 - protein, 60 aa, synthetic construct
  source 1..60: note = fuGFP
VSSGEDIFSGLVPILIELEGDVNGHRFSVRGEGYGDASNGKLEIKFICTTGRLPVPWPTL

SEQ ID NO: 13 - protein, 60 aa, synthetic construct
  source 1..60: note = Wild Type GFP
MSKGEELFTGVVPILVELDGDVNGHKFSVSGEGEGDATYGKLTLKFICTTGKLPVPWPTL

SEQ ID NO: 14 - protein, 60 aa, synthetic construct
  source 1..60: note = fuGFP
VTTLSYGVQCFAKYPEHMRQNDFFKSAMPDGYVQERTISFKEDGTYKTRAEVKFEGEALV

SEQ ID NO: 15 - protein, 60 aa, synthetic construct
  source 1..60: note = wild-type GFP
VTTFSYGVQCFSRYPDHMKQHDFFKSAMPEGYVQERTIFFKDDGNYKTRAEVKFEGDTLV

SEQ ID NO: 16 - protein, 60 aa, synthetic construct
  source 1..60: note = fuGFP
NRIDLKGLEFKEDGNILGHKLEYSFNSHYVYITADKNRNGLEAQFRIRHNVDDGSVQLAD

SEQ ID NO: 17 - protein, 60 aa, synthetic construct
  source 1..60: note = wild-type GFP
NRIELKGIDFKEDGNILGHKLEYNYNSHNVYIMADKQKNGIKVNFKIRHNIEDGSVQLAD

SEQ ID NO: 18 - protein, 58 aa, synthetic construct
  source 1..58: note = fuGFP
HYQQNTPIGEGPVLLPEQHYLTTNSVLSKDPQERRDHMVLVEFVTAAGLSLGMDELYK

SEQ ID NO: 19 - protein, 58 aa, synthetic construct
  source 1..58: note = wild-type GFP
HYQQNTPIGDGPVLLPDNHYLSTQSALSKDPNEKRDHMVLLEFVTAAGITHGMDELYK

SEQ ID NO: 20 - DNA, 36 bp, synthetic construct
  source 1..36: mol_type = other DNA
tgcacagaatacgacaaggtcgtcaccaaggtcggc

SEQ ID NO: 21 - DNA, 30 bp, synthetic construct
  source 1..30: mol_type = other DNA; note = DNA Amp2F
ctggatctcaacagcggtaagatccttgag

SEQ ID NO: 22 - DNA, 30 bp, synthetic construct; identical to SEQ ID NO: 21
  source 1..30: mol_type = other DNA; note = DNA primer

SEQ ID NO: 23 - DNA, 30 bp, synthetic construct
  source 1..30: mol_type = other DNA; note = DNA Amp1R primer
gttgagatccagttcgatgtaacccactcg

SEQ ID NO: 24 - DNA, 36 bp, synthetic construct
  source 1..36: mol_type = other DNA; note = DNA primer
tgcacgccatacgacaaggtcgtcaccaaggtcggc

SEQ ID NO: 25 - DNA, 30 bp, synthetic construct; identical to SEQ ID NO: 21
  source 1..30: mol_type = other DNA; note = DNA Amp2F primer

SEQ ID NO: 26 - DNA, 32 bp, synthetic construct
  source 1..32: mol_type = other DNA; note = DNA primer
cgtatggcgtgcagtgttttgcgaagtatccg

SEQ ID NO: 27 - DNA, 30 bp, synthetic construct; identical to SEQ ID NO: 23
  source 1..30: mol_type = other DNA; note = DNA Amp1R primer; note = DNA Amp1R primer

(19) United States
(12) Patent Application Publication
(10) Pub. No.: US 2025/0075220 A1
(43) Pub. Date: Mar. 6, 2025

(54) Method of Multi-Site Directed Mutagenesis on Plasmids Using PCR

(71) Applicant: Zhenyu Zhu, Lynnfield, MA (US)

(72) Inventor: Zhenyu Zhu, Lynnfield, MA (US)

(21) Appl. No.: 18/441,603

(22) Filed: Feb. 14, 2024

Related U.S. Application Data
(60) Provisional application No. 63/536,481, filed on Sep. 4, 2023.

(51) Int. Cl. C12N 15/64

(52) U.S. Cl. CPC C12N 15/64

(57) ABSTRACT

An improved method of site directed mutagenesis on plasmids by PCR is described, where at least two mutations are added to the parts of the plasmid DNA by site direct mutagenesis. One mutation is an intended mutation, and the other mutation changes the function of the mutated plasmid from the function of the template plasmid. The functional change is one to allow reduction of the retention of non-integrated template DNA, even in the absence of wild type template digestion with DpnI.

### BACKGROUND

Site directed mutagenesis (SDM) is a technique to change a DNA sequence in a directed, non-random manner, including by replacement, deletion, insertion, or a mix of all such changes. In SDM, one first synthesizes a DNA primer with a desired mutation, and where the remainder of the DNA primer is complementary to the template DNA around the mutation site, so it can hybridize with the DNA in the gene of interest. The mutation may be a single base change (a point mutation), multiple base changes, deletions, or insertions. The single-strand primer is then extended to copy the rest of the gene, using a DNA polymerase. The gene thus copied contains the mutated site, and it can then be introduced into a host cell using a vector and cloned. Mutants can be selected by DNA sequencing to confirm that they contain the desired mutation. Plasmids are a common target for site directed mutagenesis.

A recent paper (1) has a summary of the history of the PCR based SDM, shown below as Table 1:

TABLE 1
PCR based SDM

| Method | Method name | steps |
|---|---|---|
| Type A | Overlapping extension PCR and subcloning | Overlapping PCR, blunt end |
| | | ligation, then transform |
| Type B | Whole-plasmid PCR amplification with a pair of | Inverse PCR, DpnI digestion |
| | complementary mutagenic primers | and transformation |
| Type C | Whole-plasmid PCR, phosphorylation, and | PCR and phosphorylation, |
| | ligation | ligation, then transformation |
| Type D | Dividing the entire plasmid by two or more PCR | PCR, Exonuclease, Gibson |
| | reactions and assembling the plasmid in vitro | assembly, or golden gate, then |
| | | transform |
| Type E | Two pairs of complementary primers, PCR, and | PCR, DpnI/digestion, and |
| | direct transformation | transformation |

The methods above can be combined, e.g., Type C, where the primers are not complementary, can be performed using two or more pairs of primers where the PCR products are phosphorylated and ligated, followed by cell transformation. Type E also can employ more than two pairs of primers.

If the starting template plasmid is, e.g., N6A, which is fully methylated, the restriction enzyme DpnI, which digests methylated DNA at Gm⁶ATC, can be conveniently used to remove methylated template DNA and leave the unmethylated DNA products from PCR unaffected. Such use of DpnI has been a critical step in the mutagenesis Types B, C, D and E above. It is especially important in Types B and E. However, DpnI is known to digest hemi-methylated DNA, which is the product from the first round of PCR, very slowly (2). The remaining template is unchanged (methylated wild type). In addition to issues with digesting hemi-methylated DNA, DpnI can lose activity during storage and is not especially compatible with certain polymerase buffers. Any of these circumstances can significantly increase the quantity of wild type in the final mixture.

Thus, what is needed is a more effective way to decrease wild type in the final mixture, to leave substantially only unmethylated DNA products from PCR in the final mixture.

### SUMMARY

In this invention, at least one functional switch (e.g., adding a selection marker) and at least one added mutation are simultaneously implemented at the stage of re-combination of two PCR products, following site directed mutagenesis (“SDM”) and PCR using primer pairs for the functional switch and the added mutation, on plasmid DNA. Following PCR, one (or more) of the plasmid DNA sections includes the mutation for the functional switch(es), and one (or more) of the plasmid DNA sections is for the added mutation(s). The PCR of the different plasmid DNA sections is done using different primer pairs in different reactions, followed by recombination of the PCR products and then transformation of a target cell with the PCR products. The recombination can be by ligation or mixing of the PCR products. Recombination is then followed by selecting for the functional change in the transformed cells. Two examples of switching the fluorescence of fuGFP back and forth are described below in detail.

This invention is especially useful when polynucleotides with targeted mutations are sought for in a plasmid, including such immediately useful mutations as protein engineering mutations, restriction enzymes, or mutations encoding a sought-after protein. The functional switch with at least one added mutation, where both the functional switch and the mutation are added by site directed mutagenesis, is described in further detail with examples, below. The technique can be applied conveniently, in either bacterial or eukaryotic cells, to make large amounts of DNA that codes for any protein of interest; or, itself has a sequence of interest. See generally U.S. Pat. No. 5,556,747 (incorporated by reference) for additional SDM background.

### BRIEF DESCRIPTION OF THE FIGURES

FIGS. 1A to 1C illustrate the stages of Ampicillin resistance recovery. FIG. 1A shows the structure of the starting template plasmid. FIG. 1B illustrates PCR with primers from the Ampicillin resistance gene. FIG. 1C illustrates the structure of the assembly mutated plasmid.

FIGS. 2A to 2C illustrate the stages of simultaneous site directed mutagenesis and changing Ampicillin resistance. FIG. 1A illustrates the starting template plasmid. FIG. 1B illustrates there are two PCRs each with a different primer pair, from the site directed mutagenesis and the Ampicillin resistance gene; the dotted and dashed line illustrate the two separate PCR products; FIG. 1C illustrates the mutated plasmids with the site directed mutation and the Ampicillin resistance recovery gene.

FIGS. 3A to 3D illustrate the plasmids used in the mutagenesis, where the star in FIG. 3B indicates a Serine residue at amino acid position 68 in NHfuGFP. FIG. 1A illustrates the structure of the plasmid pKaLNHfuGFPWT. FIG. 1B illustrates the structure of the plasmid pALNHfuGFPG68S.

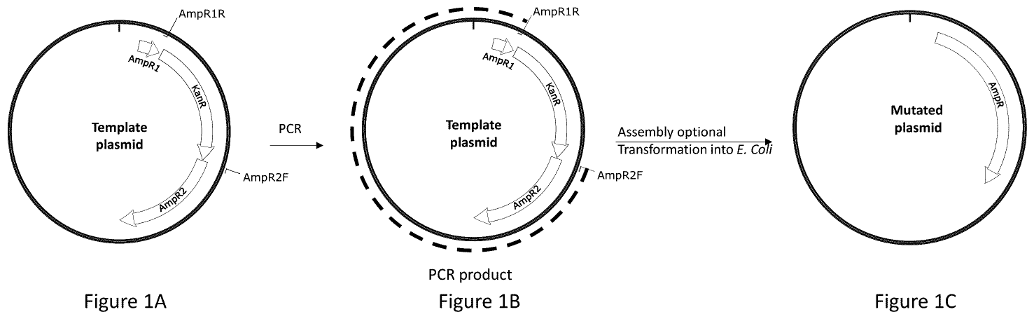

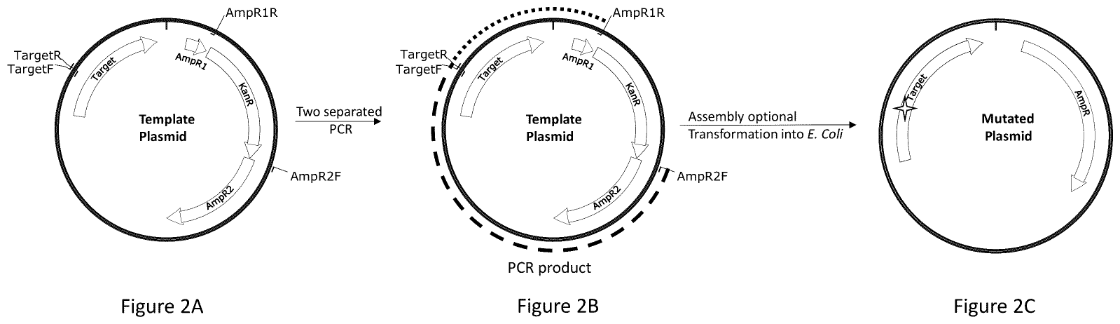

FIG. 1C illustrates the structure of the plasmid pKALNHfuGFPG68S. FIG. 1D illustrates the structure of the plasmid pALNHfuGFP wild type.

FIG. 4A shows a picture of the mutagenesis product from plasmid pKaLNHfuGFPWT using PCR, where the products are plated on agarose gel following the first and second PCR reactions.

FIG. 4B shows a picture of an agar plate of the resulting mutagenesis colony following transformation of cells with the products in FIG. 4A.

FIG. 5A shows a picture of the mutagenesis product from plasmid pKaLNHfuGFPG68S using PCR, where the products are plated on agarose gel following the first and second PCR reactions.

FIG. 5B shows a picture of an agar plate of the resulting mutagenesis colony following transformation of cells with the products in FIG. 5A.

### DETAILED DESCRIPTION

In the present invention, site directed mutagenesis is used to change the function of the mutants generated by PCR from the template plasmid, and, to change one or more additional sites on the mutant. This allows eliminating or distinguishing the mutagenesis/PCR products from the template plasmid, without using DpnI, after transformation of a cell with the mutant plasmid, by selecting for the functional change in the transformed cells. These two-PCRs (or more, to add more mutations using added primer pairs) followed by selection described herein, can be used to isolate large amounts of any mutant DNA of interest.

As an example, site directed mutagenesis is used in two separate PCR reactions, where one PCR is with the primer for the original site directed mutagenesis (“SDM”), and the other PCR is with the primers directed to a sequence region which can make a functional change, i.e., adding a selection marker, in the product. The mutation method described herein works whether the mutagenesis is type C, D or E in Table 1 above. Some of the products of this mutation method, and their properties and how to use the properties in selection, are listed in Table 2. More than one such functional change can be created in the PCR product, using the techniques of the invention, by using an additional primer with the added functional change, or by using the same primer with an additional difference representing an added functional change, from the template plasmid DNA.

TABLE 2
Simultaneous functional changes with SDM

| | | Properties of the |
|---|---|---|
| | | transformed E. Coli with |
| | Possible list of the function changes | the mutated plasmid |
| | in the transformed product, | (which the template |
| | different from the product with the | plasmid E. Coli cannot |
| Function Type | template plasmid | perform) |
| Antibiotic resistance | A new type of resistance | Grow on the new resistance |
| change | | plate |
| | Increase antibiotic resistance by | Grow in the higher |
| | enhance the expression of the | concentration of the |
| | antibiotic resistance gene | antibiotic |
| Cell growth | Template plasmid expresses DpnI, | Grow in the Dam⁺ E. Coli |
| conditions change | one primer mutation is inactivation | strain |
| | of DpnI activity | |
| | Template plasmid expresses a Dcm | Grow in the Dcm⁺ E. Coli |
| | specific enzyme | strain |
| | Template plasmid expresses a | Grow without the |
| | restriction enzyme | expression of methylase |
| | Template plasmid expresses | Grow in a strain in which |
| | methylase | methylation is toxic |
| | Template plasmid expresses a | Grow after the toxic gene is |
| | conditionally toxic gene | removed in PCR |
| | Template plasmid has a conditional | Grow in a strain with an |
| | high strength promoter | RNA polymerase such as T7 |
| | | promoter |
| | Template plasmid has a temperature | Grow in the previous |
| | sensitive replication origin | inhibition temperature |
| | Template plasmid makes cell growth | Normal growth on plate or |
| | slower | in culture |
| Cell appearance | Template plasmids or mutated | Mutants show changed |
| change | plasmids express indication genes, | color, template has original |
| | such GFP | color |
| | Template plasmids or mutated | Mutants will not be blue in |
| | plasmids express indicating enzyme | the presence of X-Gal |
| | such as beta-galactosidase | |
| | Template plasmids or mutated | Mutants grow normally |
| | plasmids express agarase | while the template plasmids |
| | | make a pit on an agar plate |

The cells transformed in Table 2 can be any competent cells for transformation and cloning. More specifically, the plasmids (template or mutated) can be expressed in any suitable host system, including a bacterial, yeast, fungal, baculovirus, plant or mammalian host cell. For bacterial host cells, suitable promoters for directing transcription of the nucleic acid constructs of the present disclosure, include the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (Villa-Kamaroff et al., A bacterial clone synthesizing proinsulin (1978), Proc. Natl Acad. Sci. USA 75:3727-3731), as well as the tac promoter (DeBoer et al., The tac promoter: a functional hybrid derived from the trp and lac promoters (1983), Proc. Natl Acad. Sci. USA 80:21-25).

For filamentous fungal host cells, suitable promoters for directing the transcription of the nucleic acid constructs of the present disclosure include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof.

In a yeast host, useful promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are described by Romanos et al., Foreign gene expression in yeast: a review (1992), Yeast 8:423-488.

For baculovirus expression, insect cell lines derived from Lepidopterans (moths and butterflies), such as Spodoptera frugiperda, are used as host. Gene expression is under the control of a strong promoter, e.g., pPolh.

Plant expression vectors are based on the Ti plasmid of Agrobacterium tumefaciens, or on the tobacco mosaic virus (TMV), potato virus X, or the cowpea mosaic virus. A commonly used constitutive promoter in plant expression vectors is the cauliflower mosaic virus (CaMV) 35S promoter.

For mammalian expression, cultured mammalian cell lines such as the Chinese hamster ovary (CHO), COS, including human cell lines such as HEK and HeLa may be used to produce the tagged restriction enzyme. Examples of mammalian expression vectors include the adenoviral vectors, the pSV and the pCMV series of plasmid vectors, vaccinia and retroviral vectors, as well as baculovirus. The promoters for cytomegalovirus (CMV) and SV40 are commonly used in mammalian expression vectors to drive gene expression. Non-viral promoters, such as the elongation factor (EF)-1 promoter, are also known.

The control sequence for the expression may also be a suitable transcription terminator sequence, that is, a sequence recognized by a host cell to terminate transcription. The terminator sequence is operably linked to the 3′ terminus of the nucleic acid sequence encoding the polypeptide. Any terminator which is functional in the host cell of choice may be used.

For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Terminators for insect, plant and mammalian host cells are also well known.

The control sequence may also be a suitable leader sequence, that is, a nontranslated region of an mRNA that is important for translation by the host cell. The leader sequence is operably linked to the 5′ terminus of the nucleic acid sequence. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells can be from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

The control sequence may also be a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. The foreign signal peptide coding region may be required where the coding sequence does not naturally contain a signal peptide coding region.

Alternatively, the foreign signal peptide coding region may simply replace the natural signal peptide coding region in order to enhance secretion of the polypeptide. However, any signal peptide coding region which directs the expressed polypeptide into the secretory pathway of a host cell of choice may be used.

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, Protein secretion in Bacillus species (1993), Microbiol Rev 57:109-137.

Effective signal peptide coding regions for filamentous fungal host cells can be the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase.

Useful signal peptides for yeast host cells can be from the genes for Saccharomyces cerevisiae alpha-factor and Saccharomyces cerevisiae invertase. Signal peptides for other host cell systems are also well known.

The control sequence may also be a propeptide coding region that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (WO 95/33836, incorporated by reference).

Where both signal peptide and propeptide regions are present at the amino terminus of a polypeptide, the propeptide region is positioned next to the amino terminus of a polypeptide and the signal peptide region is positioned next to the amino terminus of the propeptide region.

It may also be desirable to add regulatory sequences, which allow the regulation of the expression of the tagged restriction enzyme relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include the lac, tac, and trp operator systems. In yeast host cells, suitable regulatory systems include, as examples, the ADH2 system or GAL1 system. In filamentous fungi, suitable regulatory sequences include the TAKA alpha-amylase promoter, Aspergillus niger glucoamylase promoter, and Aspergillus oryzae glucoamylase promoter. Regulatory systems for other host cells are also well known.

Other examples of regulatory sequences are those which allow for gene amplification. In eukaryotic systems, these include the dihydrofolate reductase gene, which is amplified in the presence of methotrexate, and the metallothionein genes, which are amplified with heavy metals. In these cases, the nucleic acid sequence would be operably linked with the regulatory sequence.

Another embodiment includes a recombinant expression vector comprising a polynucleotide encoding an engineered tagged restriction enzyme or a variant thereof, and one or more expression regulating regions such as a promoter and a terminator, and a replication origin, depending on the type of hosts into which they are to be introduced. The various nucleic acid and control sequences described above may be joined together to produce a recombinant expression vector which may include one or more convenient restriction sites to allow for insertion or substitution of the nucleic acid sequence encoding the tagged restriction enzyme at such sites. Alternatively, the nucleic acid sequences of the tagged restriction enzyme may be expressed by inserting the nucleic acid sequences or a nucleic acid construct comprising the sequences into an appropriate vector for expression. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the tagged restriction enzyme polynucleotide sequence. The choice of the vector will typically depend on the compatibility of the vector with the host cell into which the vector is to be introduced. The vectors may be linear or closed circular plasmids.

The expression vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The expression vector herein preferably contains one or more selectable markers, which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers, which confer antibiotic resistance such as ampicillin, kanamycin, chloramphenicol (Example 1) or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus. Selectable markers for insect, plant and mammalian cells are also well known.

The expression vectors of the present invention preferably contain an element(s) that permits integration of the vector into the host cell's genome or autonomous replication of the vector in the cell independent of the genome. For integration into the host cell genome, the vector may rely on the nucleic acid sequence encoding the polypeptide or any other element of the vector for integration of the vector into the genome by homologous or nonhomologous recombination.

Alternatively, the expression vector may contain additional nucleic acid sequences for directing integration by homologous recombination into the genome of the host cell. The additional nucleic acid sequences enable the vector to be integrated into the host cell genome at a precise location(s) in the chromosome(s). The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are P15A ori, or the origins of replication of plasmids pBR322, pUC19, pACYC177 (which plasmid has the P15A ori), or pACYC184 permitting replication in E. coli, and pUB110, pE194, pTA1060, or pAM31 permitting replication in Bacillus. Examples of origins of replication for use in a yeast host cell are the 2 micron origins of replication, ARS1, ARS4, the combination of ARS1 and CEN3, and the combination of ARS4 and CEN6. The origin of replication may be one having a mutation which makes it's functioning temperature-sensitive in the host cell (see, e.g., Ehrlich, Replication of plasmids from Staphylococcus aureus in Escherichia coli (1978), Proc Natl Acad Sci. USA 75:1433).

More than one copy of a nucleic acid sequence of the tagged restriction enzyme may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

Expression vectors are commercially available. Suitable commercial expression vectors include p3×FLAG™ expression vectors from Sigma-Aldrich Chemicals, St. Louis Mo., which includes a CMV promoter and hGH polyadenylation site for expression in mammalian host cells and a pBR322 origin of replication and ampicillin resistance markers for amplification in E. coli. Other suitable expression vectors are pBluescriptII SK(-) and pBK-CMV, which are commercially available from Stratagene, LaJolla Calif., and plasmids which are derived from pBR322 (Gibco BRL), pUC (Gibco BRL), pREP4, pCEP4 (Invitrogen) or pPoly (Lathe et al., Plasmid and bacteriophage vectors for excision of intact inserts (1987) Gene 57:193-201.

Suitable host cells for expression of a polynucleotide encoding the tagged restriction enzyme, are well known in the art and include but are not limited to, bacterial cells, such as E. coli, Lactobacillus kefir, Lactobacillus brevis, Lactobacillus minor, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, BHK, 293, and Bowes melanoma cells; and plant cells. Appropriate culture mediums and growth conditions for the above-described host cells are well known in the art.

Polynucleotides for expression may be introduced into cells by various methods known in the art. Techniques include among others, electroporation, biolistic particle bombardment, liposome mediated transfection, calcium chloride transfection, and protoplast fusion. Various methods for introducing polynucleotides into cells are known to the skilled artisan.

Engineered peptides expressed in a host cell can be recovered from the cells and or the culture medium using any one or more of the well-known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available under the trade name CelLytic B.TM. from Sigma-Aldrich of St. Louis Mo.

Chromatographic techniques for isolation of the engineered peptides include, among others, reverse phase chromatography high performance liquid chromatography, ion exchange chromatography, gel electrophoresis, and affinity chromatography. Conditions for purification will depend, in part, on factors such as net charge, hydrophobicity, hydrophilicity, molecular weight, molecular shape, and will be apparent to those having skill in the art.

In some embodiments, affinity techniques may be used to isolate the engineered peptides. For affinity chromatography purification, any antibody which specifically binds the engineered peptides may be used. For the production of antibodies, various host animals, including but not limited to rabbits, mice, rats, etc., may be immunized by injection with a compound. The compound may be attached to a suitable carrier, such as BSA, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette Guerin) and Corynebacterium parvum.

Examples of the methods of the invention applied to produce desired mutants, follows.

### Examples

### Example 1: fuGFP Wild Type (WT) and G68S Mutant

fuGFP is a variant of Green Fluorescent Protein (3) which, under the excitation of long wavelength UV light, appears to be green. Also, fuGFP has a much higher thermostability than GFP at 37° C., which makes the colony express fuGFP more easily when grown overnight at 37° C. The DNA sequence (SEQ ID NO:1) is translated into the protein (SEQ ID NO:2). The fuGFP has a much different protein from GFP WT (4) (SEQ ID NO:3), where the comparison is as follows (identical residues in each sequence are bolded below, and even numbered sequences [12, 14, 16, 18] are fuGFP and odd numbered sequences [13, 15, 17, 19] are wild-type GFP):

| 1 | VSSGEDIFSGLVPILIELEGDVNGHRFSVRGEGYGDASNGKLEIKFICTTGRLPVPWPTL | 61 |
|---|---|---|
| | (SEQ ID NO: 12) | |
| 1 | MSKGEELFTGVVPILVELDGDVNGHKFSVSGEGEGDATYGKLTLKFICTTGKLPVPWPTL | 60 |
| | (SEQ ID NO: 13) | |
| Query62 | | |
| 62 | VTTLSYGVQCFAKYPEHMRQNDFFKSAMPDGYVQERTISFKEDGTYKTRAEVKFEGEALV | 121 |
| | (SEQ ID NO: 14) | |
| 61 | VTTFSYGVQCFSRYPDHMKQHDFFKSAMPEGYVQERTIFFKDDGNYKTRAEVKFEGDTLV | 120 |
| | (SEQ ID NO: 15) | |
| 122 | NRIDLKGLEFKEDGNILGHKLEYSFNSHYVYITADKNRNGLEAQFRIRHNVDDGSVQLAD | 181 |
| | (SEQ ID NO: 16) | |
| 121 | NRIELKGIDFKEDGNILGHKLEYNYNSHNVYIMADKQKNGIKVNFKIRHNIEDGSVQLAD | 180 |
| | (SEQ ID NO: 17) | |
| 182 | HYQQNTPIGEGPVLLPEQHYLTTNSVLSKDPQERRDHMVLVEFVTAAGLSLGMDELYK | 239 |
| | (SEQ ID NO: 18) | |
| 181 | HYQQNTPIGDGPVLLPDNHYLSTQSALSKDPNEKRDHMVLLEFVTAAGITHGMDELYK | 238 |
| | (SEQ ID NO: 19) | |

One of the GFP mutants, G67S, is found to lose green fluorescence (5), and this mutation is conserved in fuGFP, which has one position shifted from wild-type GFP. It is reasonable to assume, therefore, that G68S fuGFP also loses green fluorescence. On the other hand, when the residue at 68 in fuGFP is mutated back to Gly (G), the florescence is restored.

### Examples 2 and 3 (Introduction)

When an additional tag for purification is added to the target protein, the amino acid numbers from the N-terminus are represented as if there is no tag. In the following examples Nos. 2 & 3, there is an additional N-terminal 6×His tag added to the fuGFP (designated, “NHfuGFP”). The DNA sequence of NHfuGFP WT is in SEQ ID NO:4, and the protein sequence of NHfuGFP WT is SEQ ID NO:5. The DNA sequence of NHfuGFP G68S is SEQ ID NO:6, and the protein sequence of NHfuGFP G68S is SEQ ID NO:7.

### Example 2: Construction of a Template for Antibiotic Resistance Switch

Antibiotic resistance switch, from the template to the mutant, is a relatively simple functional change to initiate. In this construction (FIG. 1), Kanamycin resistance gene (KanR) is in the middle of an Ampicillin resistance gene (AmpR), which is divided into two parts: AmpR1 and AmpR2. When a deletion mutation is performed to remove the Kanamycin resistance gene, the Ampicillin resistance gene is nevertheless retained. The primers for the deletion mutation can be fully or partially overlapped, or not overlapped if using post-PCR phosphorylation and ligation.

The primer pairs for the deletion mutation are named AmpR1R and AmpR2F. When overlapped primers are used, the Ampicillin resistance gene is recovered after cell transformation, and all transformed cells (E. Coli) without the mutated plasmids also lack the Ampicillin resistance gene, and thus cannot grow on agar plates with ampicillin. The transformation can be direct or after the reaction, e.g., when using cloning kits such as ZY Cloning method kits (ZyCloning, Woburn, MA). If no overlapping primers are used, but the protocol uses post-PCR phosphorylation and ligation, the plasmids with the Ampicillin resistance gene are recovered in vitro. However, the resistance to Ampicillin still needs to be assessed and confirmed after the transformation into cells.

When another targeted mutagenesis is performed on the same plasmid, whether it is for some specific gene or sequence editing, the mutation primers on the Ampicillin resistance gene are the same, i.e., fully or partially overlapping, or not overlapping, if followed by phosphorylation and ligation. In the invention, using one primer pair for each target site and one primer pair for the ampicillin resistance recovery site, the two PCRs are performed separately. After both PCRs are complete, either by mixing equal volumes without assembly, or following in vitro assembly, the PCR products are transformed into competent cells, like E. Coli. No DpnI digestion is necessary as little wild type is present.

The transformed E. Coli is plated on an agar plate with Ampicillin (preferably, 100 μg/ml). The transformed cells will have Ampicillin resistance and their colonies will appear on the agar plate.

### Example 3: The Feature of Template Plasmid pKaLNHfuGFPWT and pKaLNHfuGFPG68S

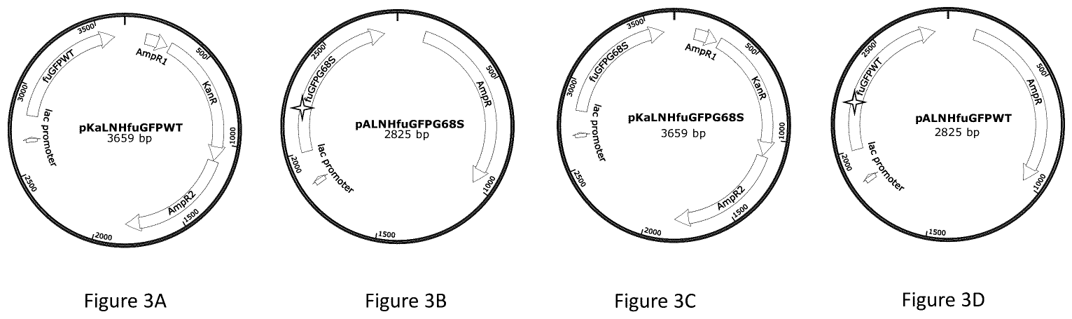

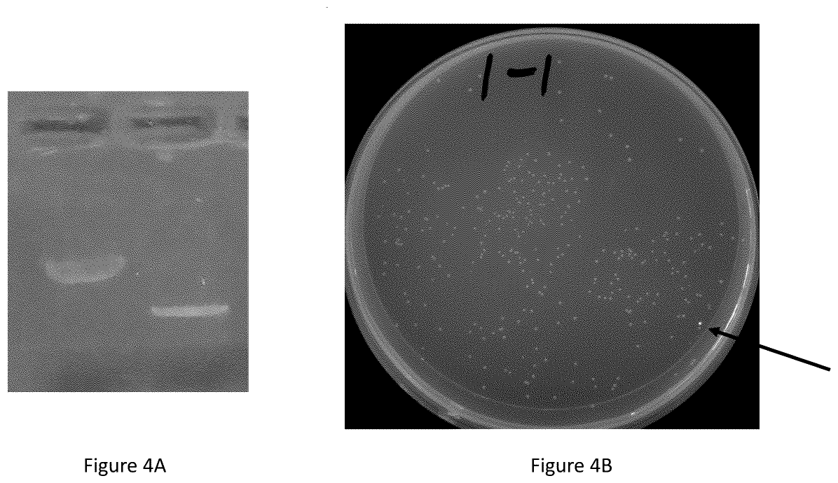

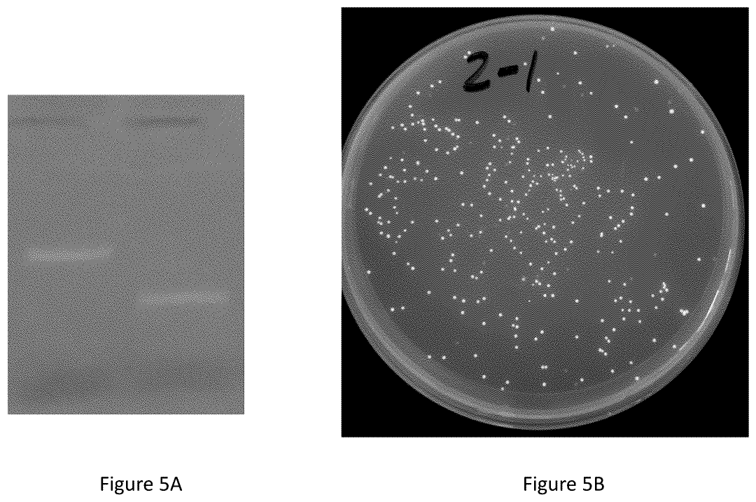

The complete pKaLNHfuGFPWT sequence is in SEQ ID NO:8. It features hybrid antibiotic resistance with a Kanamycin resistance gene in two parts of the Ampicillin resistance gene fuGFP is under the constitutive expression promoter plac. (FIG. 3) So, the E. Coli colony with pKaLNHfuGFPWT is green when exposed to long wavelength UV light, which shows as bright spots in the gray scale picture. The mutated product with the Kanamycin resistance gene deletion and the G68S mutation, is pALNHfuGFPG68S (SEQ ID NO:9). The colony on the plate with this plasmid is not fluorescent when exposed to UV light and appears as pale gray spots in the gray scale picture in FIG. 4.

The complete sequence of pKaLNHfuGFPG68S is SEQ ID NO:10, and the E. Coli colony with this plasmid has no Ampicillin resistance and is not fluorescent under UV and appears as pale gray spots in the gray scale picture in FIG. 4. The mutated product from the Kanamycin resistance gene deletion with the S68G mutation back, is pALNHfuGFPWT (SEQ ID NO:11). The colony with this product plasmid is fluorescent green under UV light and shows as bright white spots in the gray scale picture in FIG. 4.

### Example 4: Mutagenesis of pKaLNHfuGFPWT to pALNHfuGFPG68S with the Simultaneous Change of Kanamycin Resistance Gene to Ampicillin Resistance Gene and NHfuGFPWT to NHfuGFPG68S

Two separate PCR reactions are set up with the following primers:
For PCR reaction 1:

G68SR:
(SEQ ID NO: 20)
5′ TGCACAGAATACGACAAGGTCGTCACCAAGGTCGGC 3′
Amp2F:
(SEQ ID NO: 21)
5′ CTGGATCTCAACAGCGGTAAGATCCTTGAG 3′

For PCR reaction 2:

G68SF:
(SEQ ID NO: 22)
5′ CGTATTCTGTGCAGTGTTTTGCGAAGTATCCG 3′
Amp1R:
(SEQ ID NO: 23)
5′ GTTGAGATCCAGTTCGATGTAACCCACTCG 3′

G68SF and G68SR have a 13 bp homology as underlined, and Amp1R and Amp2F have a 12 bp homology as double underlined.
Make a primer mix of 0.5 μM forward and reverse primers.
Reaction reagents are as follows:
4 μl of 0.5 μM primer mix;
1 μl of 3.7 ng template plasmid pKaLNHfuGFPWT;
5 μl of Q5 High-Fidelity 2× Master Mix (New England Biolabs, Ipswich, MA);
The total volume is 10 μl.
The PCR reaction is performed as follows:
98° C. 30 sec;
Then followed by 28 cycles of:
   98° C. 5 sec;
   55° C. 10 sec; and
   72° C. 1 min.
Then followed by a 72° C. for 2 min, followed by a cool down period.

2 μl of PCR product was run on 0.8% agarose gel under 200 v for 20 minutes. (FIG. 4). 1 μl of PCR product from each PCR was mixed, then transformed into 50 μl DH10B with enhancer (ZyCloning, Woburn, MA), and then immediately plated on an agar plate with Ampicillin. The plate was grown overnight at 37° C. After checking with UV light, out of a total 289 colonies, 288 were pale gray and 1 was bright white, as indicated by the arrow in FIG. 4.

Even without treatment with DpnI, the WT remaining is only 0.3% of the total, meaning that this antibiotic switch method leaves very little remaining WT. In prior methods, lack of DpnI digestion will result in significant false positives, which are hard to find unless sequenced. In this method, however, false positives are maximumly reduced. The sole remaining WT colony on the plate could be a result of transformation of the template plasmid and PCR fragments, where the E. Coli had plasmids of both template and mutated product, such that a template WT colony remained on the plate. There is also a small chance that the ampicillin resistance results from other causes, such as cellular mutation of E. Coli rather than transformation.

Not all the pale gray products in FIG. 4 are necessarily the correctly mutated product. In the assembly in vitro, there is a chance that the assembly had mistakes like deletions, insertions, double insertions through homology, and mutations during PCR. But this example nevertheless shows that the remaining template is present only at a very low level.

### Example 5: Mutagenesis of pKaLNHfuGFPG68S to PALNHfuGFPWT with the Simultaneous Change of Kanamycin Resistance Gene to Ampicillin Resistance Gene and NHfuGFPG68S to NHfuGFPWT

Two separate PCR reactions are set up with the following formulation:
For PCR reaction 1

S68GR:
(SEQ ID NO: 24)
5′ TGCACGCCATACGACAAGGTCGTCACCAAGGTCGGC 3′
Amp2F:
(SEQ ID NO: 25)
5′ CTGGATCTCAACAGCGGTAAGATCCTTGAG 3′

For PCR reaction 2

S68GF:
(SEQ ID NO: 26)
5′ CGTATGGCGTGCAGTGTTTTGCGAAGTATCCG 3′
Amp1R:
(SEQ ID NO: 27)
5′ GTTGAGATCCAGTTCGATGTAACCCACTCG 3′

G68SF and G68SR have a 13 bp homology as underlined, and Amp1R and Amp2F have a 12 bp homology as double underlined.
Make a primer mix of 0.5 μM forward and reverse primers.
Reaction reagents are as follows:
4 μl of 0.5 μM primer mix;
1 μl of 3.7 ng template plasmid pKaLNHfuGFPS68G;
5 μl of Q5 High-Fidelity 2× Master Mix (New England Biolabs, Ipswich, MA).
The total volume is 10 μl.
PCR reaction is performed as follows:
98° C. 30 sec
Then followed by 28 cycles of:
   98° C. 5 sec;
   55° C. 10 sec; and
   72° C. 1 min.
Then followed by a 72° C. for 2 min, then followed by a cool down period.

2 μl of PCR product was run on 0.8% agarose gel under 200 v for 20 minutes. (FIG. 5). 1 μl of PCR product from each PCR was mixed, then transformed into 50 μl DH10B with enhancer (ZyCloning, Woburn, MA), and plated on agar plate with Ampicillin right away. Then the plate was grown overnight at 37° C. After checking with UV light, out of a total 335 colonies, 31 were pale gray and 304 were bright white.

In this example, the template remaining is difficult to confirm. All the remaining template colonies, with all other mistakes from in vitro assembly and PCR mutation, were 9.3% of the total colonies. The apparently correct mutation ratio was 90.7%, though it was probably a higher number in fact.

Since the PCR primers were crude grade, purified PCR primers could increase the correct mutation ratio. The mistakes of mutation and assembly are mostly in different categories, therefore, a mixture of tens of colonies would mostly represent the desired mutation, if this mutation is used in a protein function analysis.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “including”, containing “, etc. are to be read expansively and without limitation. The methods and processes illustratively described herein suitably may be practiced in differing orders of steps, and that they are not necessarily restricted to the orders of steps indicated herein or in the claims. It is also noted that as used herein and in the appended claims, the singular forms “a,” “an,” and “the” include plural reference, and the plural include singular forms, unless the context clearly dictates otherwise.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, including but not limited to Variant Sequences, and that such modifications and variations are considered to be within the scope of this invention as defined by the appended claims. without qualification or reservation expressly adopted in a responsive writing by Applicants. The invention has been described broadly and generically herein. Each of the narrower species and subgeneric groupings

### Added References (all Incorporated by Reference)

1. Yang Z., et al, A simple and economical site-directed mutagenesis method for large plasmids by direct transformation of two overlapping PCR fragments (2022) BIOTECHNIQUES VOL. 73, NO. 5, https://doi.org/10.2144/btn-2022-0085
2. Lu L., et al, Optimizing DpnI Digestion Conditions to Detect Replicated DNA (2002) BioTechniques 33:316-318
3. https://schaechter.asmblog.org/schaechter/2019/05/the-story-of-free-use-gfp-fugfp.html
4. Tsien R. Y., The green fluorescent protein. (1998) Annu. Rev. Biochem. 67:509-544.
5. Fu, G. L., GFP Loss-of-Function Mutations in Arabidopsis thaliana (2015) G3 (Bethesda). 5 (9): 1849-1855.

What is claimed is:

1. A method of site directed mutagenesis on plasmids using PCR, initiating a functional change from the template plasmid to the PCR product and adding at least one additional mutation, using at least two separate PCR reactions with, respectively, a first pair of forward and reverse primers, a second pairs of forward and reverse primers, or with additional pairs of forward and reverse primers, where a first of said PCR reactions is with the first primer pair for the additional mutation and a second PCR reaction is with the second primer pair for the functional change, comprising:
performing, in any sequence, said first and second PCR reactions on a plasmid with, respectively, the first and second primer pairs;
recombining reaction products from each PCR;
transforming a cell with the recombined products; and
using a selection method which allows location of cells with the functional change to differentiate the transformed cells with the PCR product plasmids bearing the functional change from the untransformed cells with the template plasmids.

2. The method of claim 1 wherein no DpnI is used before or after recombination.

3. The method of claim 1 wherein the second PCR reaction with the second primer pairs is performed before the first PCR reaction.

4. The method of claim 1 wherein the recombination is by ligation or mixing of the reaction products.

5. The method of claim 1 wherein the functional change is antibiotic resistance, cell growth conditions, or cell appearance, including fluorescence, or color change.

6. The method of claim 1 wherein the additional mutation(s) can be in a different region of the plasmid from the region which controls the functional change.

7. The method of claim 1 wherein more than one functional change is initiated by the same primer pair.

8. The method of claim 1 wherein more than one functional change is initiated by the different primer pairs.

9. The method of claim 1 wherein the functional change removes a Kanamycin resistance gene from the plasmid but retains the ampicillin resistance gene.

10. The method of claim 1 wherein the plasmid DNA sequence includes the sequence of SEQ ID NO: 1.

11. The method of claim 1 wherein the plasmid protein sequence includes the sequence of SEQ ID NO: 2.

12. The method of claim 11 wherein the plasmid protein has a six membered histidine tag added at its N-terminus.

13. The method of claim 12 wherein the primer pairs are SEQ ID NOS: 20 and 22; and SEQ ID NOS: 21 and 23.

14. The method of claim 12 wherein the primer pairs are SEQ ID NOS: 24 and 26; and SEQ ID NOS: 25 and 27.
